(19) United States
(12) Patent Application Publication
(10) Pub. No.: US 2023/0355447 A1
(43) Pub. Date: Nov. 9, 2023

(54) Method for wrapping a sanitary item or a stack comprising a number of sanitary items

(71) Applicant: BICMA HYGIENE TECHNOLOGIE GMBH, Mayen (DE)

(72) Inventors: Andrea ALLAR, Bendorf (DE); Heinz BARZ, Einig (DE)

(73) Assignee: BICMA HYGIENE TECHNOLOGIE GMBH, Mayen (DE)

(21) Appl. No.: 17/911,725

(22) PCT Filed: Mar. 16, 2021
(86) PCT No.: PCT/EP2021/056729

§ 371 Date: Sep. 15, 2022

(30) Foreign Application Priority Data
Mar. 16, 2020 (DE) 10 2020 203 360.9

(51) Int. Cl. A61F 13/15; A61F 13/551

(52) U.S. Cl. CPC A61F 13/15747; A61F 13/55115

(57) ABSTRACT

A method for wrapping a hygiene article or a stack of a number of hygiene articles is intended to enable them to be packaged in a wrapping formed from a sustainable raw material. For this purpose, the hygiene article(s) are fed to a wrapping station by means of a first feed unit, wherein a wrapping blank is pre-creased with folding edges suitable for forming side edges of the wrapping and is then fed to the wrapping station by means of a second feed unit, and in the wrapping station the hygiene article(s) are wrapped by folding the wrapping blank and then closing it by folding in the side flaps to form the side edges, the front and rear flaps are folded in to form the side edges, and the front and rear flaps are fixed to the side flaps and to each other.

### FIELD OF INVENTION

The invention relates to a method of wrapping a hygiene article or a stack of a number of hygiene articles. It further relates to a hygiene article provided with a wrapping.

### BACKGROUND

In the following and for the purposes of the present application, the term “hygiene product” or “hygiene article” means in particular absorbent disposable articles for absorbing body excretions. Such disposable hygiene products are usually offered and sold individually or packaged in product stacks. The hygiene products particularly considered here include, in particular, sanitary towels, nappies, incontinence products, panty liners, meat pads, pet pads (for pets), nursing pads or the like. Such products, in particular when designed as one-time or disposable products, are usually made of an absorbent material and are also provided with an absorbent core which converts absorbed liquid, for example, into a gel or the like. These products in particular have, inter alia, a comparatively soft, elastic, and deformable base body. The packaging or wrapping of such products is therefore, on the one hand, fundamentally important for hygienic reasons, but on the other hand, also desirable for functional reasons, in order to reduce the penetration of moisture to a large extent before the actual use of the hygiene article.

The packaging or wrapping of such products, individually or as product stacks with a small number of individual products, is usually carried out with regard to the requirements of the automated packaging process with a hard-wearing, elastically stretchable plastic or plastic-based film. This is usually supplied as continuous material on a roll or similar and fed to a wrapping station together with the products or product stacks to be packaged. There, a film tube surrounding the product flow is formed from the supplied film, and then the wrappings are separated, and the ends are welded so that wrappings completely enclosing the respective products are produced.

There is now a desire, inter alia for reasons of sustainability and environmental protection, to change the material of the wrapping material for such wrapped products to a sustainable, preferably biodegradable or recyclable raw material. The hygiene products themselves can be manufactured from a suitably selected, sustainable, biodegradable material, for example on a fiber basis, especially with regard to the intended design as a one-time or disposable product; for a high-quality, sustainable, and biodegradable overall ensemble, it would therefore be desirable to also suitably adapt the wrapping material to this. However, due to the process and with regard to the common, usually automated procedures in the packaging of hygiene products, the common wrapping methods are not suitable for the use of such, usually non-stretchable materials such as paper.

### BRIEF SUMMARY OF INVENTION

The invention is therefore based on the task of providing a method for wrapping a hygiene article or a stack of a number of hygiene articles, with which the packaging of the hygiene article or the stack of hygiene articles is made possible in a wrapping formed from a sustainable, in particular biodegradable and/or recyclable, raw material.

This task is solved according to the invention in that
a. the sanitary article or the stack of sanitary articles is fed to a wrapping station by means of a first feed unit,
b. a wrapping blank formed from the sustainable raw material is provided in a pre-creasing station with folding edges suitable for forming side edges or side surfaces of the wrapping and is then fed to the wrapping station by means of a second feed unit,
c. in the wrapping station, the hygiene article or the stack of hygiene articles is positioned in a predetermined desired position on the wrapping blank, and
d. in the wrapping station, the hygiene article or the stack of hygiene articles is wrapped by folding the wrapping blank and then closing it, in that
d1. the side flaps are folded to form the side edges predefined by the folding edges,
d2. the front and rear flaps are folded to form the side edges predefined by the folding edges provided for them, and
d3. the front and rear flaps are fixed to the side flaps and to one another.

Advantageous embodiments of the invention are the subject of the subclaims.

The invention is based on the consideration that a major obstacle to the conversion of the wrapping material to the desired sustainable raw materials is likely to be the process sequences of the common packaging processes for hygiene products. These common processes impose a number of requirements on the material properties of the processed packaging materials. In particular, the common processes require sufficient tear resistance and, if necessary, a certain elasticity and stretchability of the wrapping material, which may not be given for the sustainable, biodegradable materials now intended as raw materials. In order to counteract this, a fundamental process change in the packaging or wrapping of the hygiene products is therefore planned. For this purpose, elements from the field of envelope technology should be used, which, for example, enables the packaging of individualized items in paper envelopes with —comparatively high cycle and throughput rates.

Taking into account the material properties of the desired wrapping materials while keeping costs low to a large extent can be achieved in particular by producing both the goods to be wrapped and the packaging on site—in analogy to corresponding processes for enveloping goods to be shipped. For the most flexible wrapping possible that can be individually adapted, the packaging may not already be produced before it is brought together with the product to be wrapped, but only wrapping blanks can be provided first. These wrapping blanks are combined with the product or product stack, whereby especially with regard to the desired material combination of a comparatively soft product (typical for hygiene products) and a comparatively hard wrapping material (typical for paper as the desired wrapping material), the product or product stack should first be precisely positioned on the wrapping blank. Only then is the product or product stack wrapped by folding the wrapping blank.

Also with regard to the intended material combination, the wrapping blank should be suitably prepared for the subsequent folding. It should be taken into account that with regard to the expected thickness of the products or product stacks, a circumferential side edge is necessary for the wrapping. Depending on the respective specification for the end product, the products can be present and packaged either unfolded or folded, so that for this reason alone there can be different thicknesses in the product range. In order to provide these different thicknesses reliably and reproducibly, suitable folding edges should be introduced into the blank before the wrapping blank and the product are joined, which define the position and orientation of the folding edge in the manner of a predefinable target line during the subsequent folding.

The material provided for the formation of the wrapping is very preferably formed from sustainable, in particular biodegradable and/or recyclable raw material, which preferably does not produce microplastics. Paper (coated or uncoated), kraft paper, greaseproof and/or wet-strength paper, filter paper, crepe paper, glassine, eucalyptus, tissue, bamboo, papyrus, hemp, felt, wool, cotton, linen or other such materials are considered particularly suitable and thus preferred.

The material intended to form the wrapping may be single-layered or multilayered. Multi-layer material is preferably formed by a base layer laminated with a layer of other biodegradable material. The material may be smooth, pre-embossed or embossed in-line, depending on the product specification.

The material for the wrapping can have a closed or partially open surface, e.g. due to fibrous properties or due to a deliberately introduced perforation. Such a partially open surface can be particularly advantageous to ensure that the wrapping does not open again under pressure in the further course of the process due to air trapped inside the package.

Preferably, the material provided for the wrapping has a grammage or a paper weight of 25-80 gsm.

The wrapping blank can be fed into the process as a continuous web from a roll or also individually as a sheet. In the case of feeding from a continuous web, the wrapping blank is advantageously separated from the continuous web before being fed to the wrapping station, or after being combined with the product or product stack and any folding of the side flaps provided, whereby at a suitable point in the process, in a further advantageous embodiment, a crosscutting of the wrapping material between two products is provided for separation and for producing the front and rear flaps.

Advantageously, the side edges of the wrapping blank are cut to produce the side wrapping flaps before or in the wrapping station.

According to the process, it is provided that after complete wrapping of the product or product stack, i.e. after folding in all wrapping flaps, the front and rear flaps are fixed to the side flaps and/or to each other. Advantageously and for a particularly favorable and reliable process sequence, the flaps of the wrapping are thereby fixed to each other thermally and/or mechanically and/or by gluing, preferably reclosably, in or after the wrapping station. The closing of the wrapping or the fixing is particularly preferably carried out by means of adhesive fixing material, e.g. hot glue, cold glue, self-adhesive tape or the like, by means of mechanical fixing material, such as Velcro fastener, snap fastener or the like, by means of thermoplastic fixing material, e.g. hot activatable cold glue, wax, lacquer or the like, and/or without separate fixing material, e.g. by purely mechanical or thermomechanical embossing, ultrasound, insertion of the flaps into slots provided for this purpose or the like.

The fixing materials or methods used can be applied over the entire surface or in partial areas of the wrapping, for example already by the material manufacturer or inline in the machine. For this purpose, a suitable module is preferably provided in the packaging machine with which the fixing material can be suitably applied to the selected zones or partial areas, preferably by printing, spraying or other methods suitable for the material in question.

In a particularly preferred embodiment, personalization or individualization of the wrapped hygiene product or stack of hygiene products is provided or made possible by printing on the wrapper blank. The printing can be applied in the form of individual elements or ornaments or alternatively over the entire surface, on the inside and/or outside. Printing with individual elements is particularly advantageous for user or address-specific information, whereas full-surface printing can be advantageously provided, for example, to make the wrapping—opaque or non-transparent. This aspect could be particularly advantageous, for example, in the case of inherently translucent or transparent wrapping materials such as parchment or the like. Several such printings may also be provided in combination with each other.

In particular, a suitably designed printing module can be integrated into the packaging machine, with which, for example, manufacturer-specific or consumer-specific information or design elements can be applied to the wrapping.

In an alternative or additional embodiment, the envelope blank is provided with an embossing. The embossing can be in the form of a motif or alternatively as a full-surface embossing, for example to give the wrapping a desired texture and/or a special feel. Analogous to printing, this can primarily serve design purposes, for example in the form of applying an indication of origin in the manner of a seal or watermark, or also technical purposes, for example in order to effect a mechanical interlocking or connection of adjacent or superimposed parts of the wrapping by means of such an embossing.

In an alternative or additional advantageous embodiment, the wrapping blank is provided with a perforation. This can, in addition to or instead of decorative purposes, serve, for example, to specify a tear-off or tear-up edge for subsequent opening of the wrapping with a defined removal opening and/or also as a relief perforation to prevent an undesirable build-up of pressure or the undesirable formation of an air cushion inside the wrapping.

In a particularly advantageous embodiment, a window is cut out of the envelope blank, which is then sealed by gluing in a window film. The material for the window film is preferably a suitably transparent material such as parchment, for example, which harmonizes suitably with the actual wrapping material with regard to the desired sustainability aspects. Thus, even when using opaque wrapping materials such as paper, the wrapping can be designed in such a way that the user can directly inspect the product inside the wrapping and, if necessary, its condition.

Advantageously, the hygiene article or the stack of hygiene articles on the one hand and an associated wrapping blank on the other hand are fed to the wrapping station in a timed and synchronized manner. In a particularly advantageous further development, the timing or synchronization of the hygiene article or stack of hygiene articles on the one hand and the assigned wrapping blank on the other hand is monitored by interrogating a print mark, so that the correct assignment of the product to the wrapping assigned to it can be suitably monitored and the correct positioning of the product on the wrapping blank can also be ensured. The print mark can be applied at a visible or invisible position in the finished product.

In a further advantageous embodiment, the hygiene article(s) is/are fixed to the wrapping after folding. Particularly in combination with a viewing window inserted into the wrapping, this can ensure that an information area attached to or printed on the product, for example, can actually be viewed by the user and remains intact even when the wrapped product is folded or otherwise subjected to mechanical stress. Such a fixation may be particularly advantageous if the product is a sanitary napkin. Such a sanitary napkin may be provided with a silicone strip as a cover for an adhesive area, whereby the adhesive then remains on the product after this strip is removed. Therefore, in a particularly advantageous embodiment, the individual packaging of this product provides for the silicone strip to be glued or bonded to the wrapping material. This ensures that the silicone strip is automatically removed during unpacking, i.e. when the wrapping is removed, so that handling is correspondingly easier for the user. In addition, this design results in only one waste part requiring disposal. This is “unmixed” in the sense of waste or disposal management, as it is made exclusively of paper.

A wrapped hygiene article obtainable by the above-described process, in particular a wrapped elastic disposable or reusable article with integrated absorber for absorbing body fluids, provided with a wrapping made of a sustainable, in particular biodegradable and/or recyclable, raw material, is expressly considered to be independently inventive. Similarly, the use of the wrapping process for wrapping absorbent disposable articles for absorbing body excretions, in particular sanitary towels, panty liners, incontinence pads, nappies or nappy trousers for babies, adults or pets, pads as bedding, changing pads, pet pads, wound pads, or liners for food trays (e.g. meat, fish), nursing pads, disposable handkerchiefs or the like is considered to be independently inventive.

The advantages achieved with the invention consist in particular in the fact that the conceptual use of process elements used in inserting or enveloping machines in the individualized packaging of hygiene products, individually or as a stack, folded or unfolded, also makes it possible to use alternative wrapping materials, in particular sustainable, biodegradable raw materials, which would otherwise not meet the process and load requirements actually common for hygiene products. In particular, non-elastically deformable materials can be processed that are not suitable for the previous standard processes because they would wrinkle or tear there. The replacement of the commercially available plastic wrapping film for hygiene products with a more sustainable raw material, which is thus made possible, particularly aimed at and regarded as independently inventive, results in a reduction of the plastic input into the environment and a reduction of the total waste produced by using biodegradable and/or recyclable wrapping material.

If such a novel process replaces an “Easypack” process that has been common on the market to date, in which the products are laminated directly onto the wrapping film without silicone paper, this will lead to a product improvement through:
optimum adhesive glue transfer to the product, without bubble formation
omission of the critical transverse distribution of a wide adhesive glue application
omission of speed-limiting switching times with several adhesive glue strips applied transversely

### BRIEF DESCRIPTION OF THE DRAWINGS

An embodiment of the invention is explained in more detail with reference to a drawing. Shown therein is:

FIG. 1 a sanitary article immediately before it is packaged,

FIG. 2 the hygiene article according to FIG. 1 in partially packaged condition,

FIGS. 3a, 3b schematically the respective process sequence for wrapping the hygiene article according to FIG. 1, and

FIG. 4 some variants of pre-cut wrapping blanks with inserted folded edges.

Identical parts are marked with the same reference signs in all figures.

### DETAILED DESCRIPTION OF THE DRAWINGS

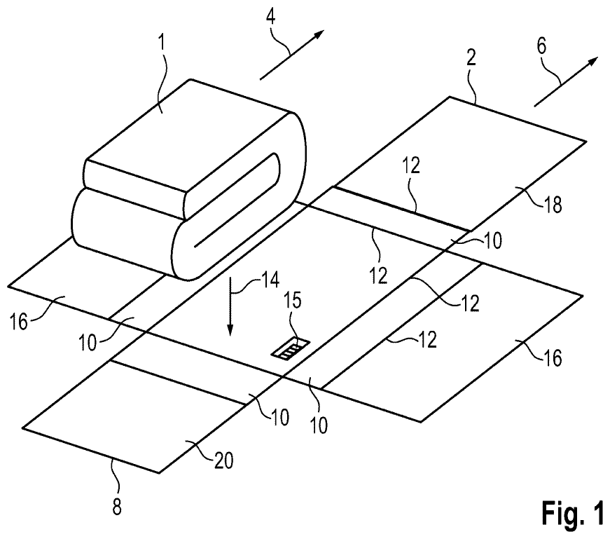

The hygiene product 1 or the hygiene article 1 according to FIG. 1, in the embodiment example an absorbent disposable article for absorbing body excretions, is to be provided with a wrapping 2 in the manner of an individual package. In the embodiment example, the hygiene product 1 is a folded product, but alternatively it may be packaged unfolded, or it could also be a stack of a number of such hygiene products 1. In particular, the hygiene product 1 may be a sanitary napkin, a nappy, an incontinence product, a panty liner, a meat pad, a pet pad, a nursing pad or the like.

For the wrapping 2, the use of a sustainable, biodegradable and/or recyclable raw material as a base material is envisaged, which preferably does not produce microplastics. Paper (coated or uncoated), kraft paper, greaseproof and/or wet-strength paper, filter paper, crepe paper, pergamide, eucalyptus, tissue, bamboo, papyrus, hemp, felt, wool, tree wool, linen or other such materials are considered to be particularly suitable and thus preferred for this purpose in the embodiment example.

Due to the material properties of such a raw material, in particular a lack of elasticity, higher brittleness and comparatively lower tear strength, the use of the usual packaging methods is not suitable for this type of hygiene article 1. In the embodiment example, it is therefore intended to wrap the hygiene article 1 in the wrapping 2 using process elements actually intended for enveloping or letter wrapping machines. For this purpose, the hygiene article 1, as sketched in FIG. 1, is first fed along a transport direction indicated by the arrow 4 to a wrapping station and there brought into overlap with a wrapping blank 8 formed from the sustainable raw material and also conveyed along the transport direction indicated by the arrow 6. Beforehand, the wrapping blank 8 was provided in a pre-creasing station with folding edges 12 suitable for forming side edges 10 of the wrapping 2.

In the wrapping station, the hygiene article 1 is positioned in a predetermined target position on the wrapping blank 8, as indicated by the arrow 14. The correct assignment of the hygiene article 1 or product to the wrapping blank assigned to it and also the correct positioning of the product on the wrapping blank 8 are suitably monitored by means of a print mark 15. The hygiene article 1 is then wrapped by folding the wrapping blank 8 and then closing it, the side flaps 16 being folded in to form the side edges 10 predefined by the folding edges 12, the front and rear flaps 18, 20 being folded in to form the side edges 10 predefined by the folding edges 12 provided for them, and finally the front and rear flaps 18, 20 being fixed to the side flaps 16 and to each other.

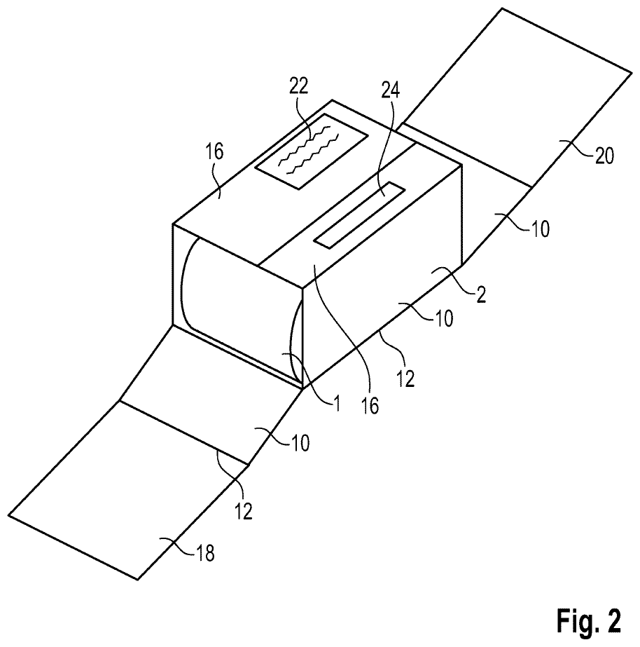

For further clarification, FIG. 2 shows the hygiene product 1 already partially surrounded by the wrapping 2; in the situation shown, the side flaps 16 have already been folded over and fixed to one another, forming the provided side edges 10 adapted to the product thickness, whereas the folding over of the front and rear flaps 18, 20 is still pending. As can also be seen from this illustration, the wrapping blank is provided with printing in the form of a motif 22 and with a window cut-out 24.

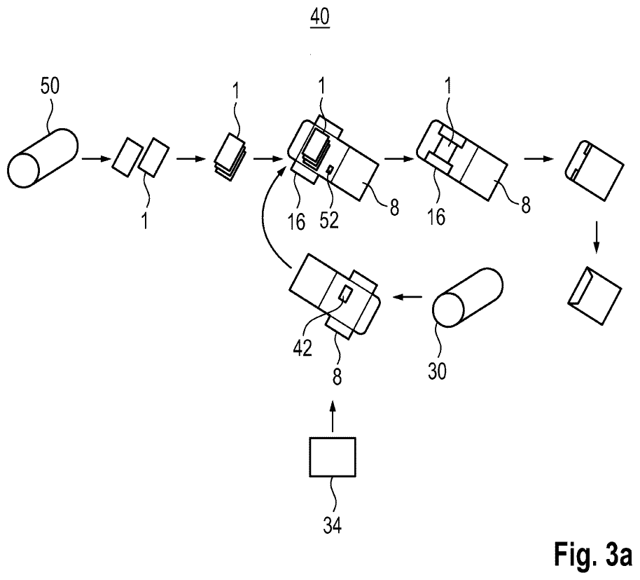

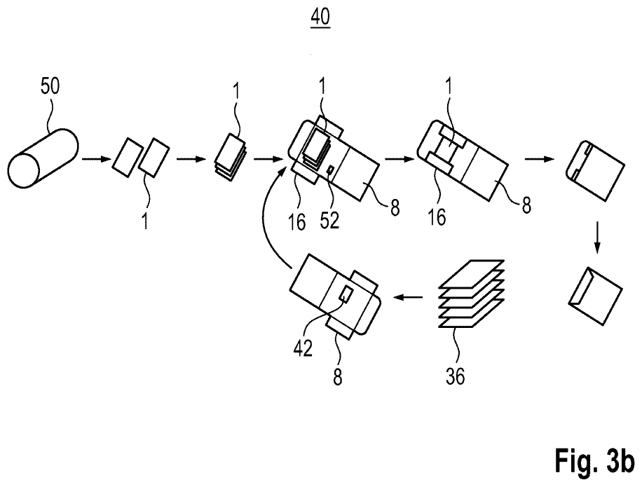

The process sequence for wrapping the hygiene product 1 is shown schematically in FIG. 3. In the first variant shown in FIG. 3a, the wrapping blanks 8 are provided via a first material web 30. In a first process step, the material web 30 is cut into the wrapping blanks 8 by means of a cutting unit 34 in the manner of a separation; alternatively, the wrapping blanks 8 according to the variant shown in FIG. 3b can also be pre-cut or provided as a single sheet or sheet. By means of the cutting unit 34, a particularly individual cutting is possible, which can also be changed from wrapping blank 8 to wrapping blank 8 by a corresponding control of the cutting unit 34. Thus, a direct and optimal adaptation to the hygiene product 1 to be wrapped is possible. If the material web 30 and thus also the wrapping blank 8 are already completely printed, the wrapping blank 8 is fed to the wrapping unit 40 in the next process step. Otherwise, the wrapping blank 8 can be individually printed in a process step not shown by a printing unit not shown before being fed to the wrapping unit 40. In addition or as an alternative, it is also possible for printing to be carried out on the material web 30 by a further printing unit. This has the advantage that printing can be carried out over the entire subsequent blank and thus no system-related print-free edges are created. By using two printing units connected one after the other, it is also possible to print on both sides of the packaging, outside and inside. Alternatively or in addition to such printing, the embossing of a motif and/or the insertion of a perforation can also be provided.

It is also possible to cut a window element 42 into the wrapping blank 8 in a further process step prior to feeding the wrapping unit 40 or together with the cutting of the wrapping blank 8 and to close it with a window film. In this way it is also possible to produce wrappings 2 with a viewing window.

The schematic representation of the process according to FIG. 3 further shows a second material web 50 for the production of the hygiene product 1 to be packaged. This second material web 50 is processed in a manner known per se, whereby the hygiene products 1 are provided by cutting, possibly folding, possibly stacking, depending on the specified requirements. The finished product 1, if necessary as a product stack, is then fed to the wrapping unit 40.

For particularly reliable and precise positioning, the hygiene product 1 on the one hand and the assigned wrapping blank 8 on the other hand are fed to the wrapping station 40 in a timed manner and synchronized with one another, the timing or synchronization of hygiene product 1 on the one hand and assigned wrapping blank 8 on the other hand being monitored by interrogating a print mark 52. When the hygiene product 1 is united with the wrapping blank 8, both are transported in a line and in synchronization with each other. This avoids relative movements between the two, and the product 1 can be placed on the wrapping blank 8 in a particularly simple manner, for example by depositing it in a precise position. Thus, the product 1 to be packaged lies in the wrapping unit 40 on the inside of the wrapping blank 8.

In a first wrapping step, the side flaps 16 of the wrapping blank 8 are first folded and wrapped around the hygiene product 1. At the same time or in a subsequent process step, the outer side of the side flaps 16 is covered with an adhesive film to enable subsequent fixing. In a second wrapping process, the front and rear flaps 18, 20 are then folded, wrapped around the product 1 and glued to the side flaps 16.

In the embodiment example, a gluing process is provided for fixation; alternatively or additionally, fixation can also be carried out by means of adhesive fixation material, e.g. hot glue, cold glue, self-adhesive tape or others, by means of mechanical fixation material, e.g. Velcro fastener, snap fastener or others, by means of thermoplastic fixation material, e.g. hot activatable cold glue, wax, varnish or others. This can be done by means of thermoplastic fixing material, e.g. hot-activated cold glue, wax, varnish or others, or without fixing material, e.g. by purely mechanical or thermomechanical embossing, ultrasound, insertion of the flaps into slots provided for this purpose or others, or by means of a combination of one or more of these possibilities.

The fusing materials can be applied over the entire surface or in partial areas, either already by the material manufacturer or inline in the machine. There, e.g. by printing, spraying, or other processes suitable for the material in question. In particular, the flaps 16, 18, 20 of the wrapping 2 can be fixed to each other thermally and/or mechanically and/or by gluing, preferably resealably, in or after the wrapping station 40.

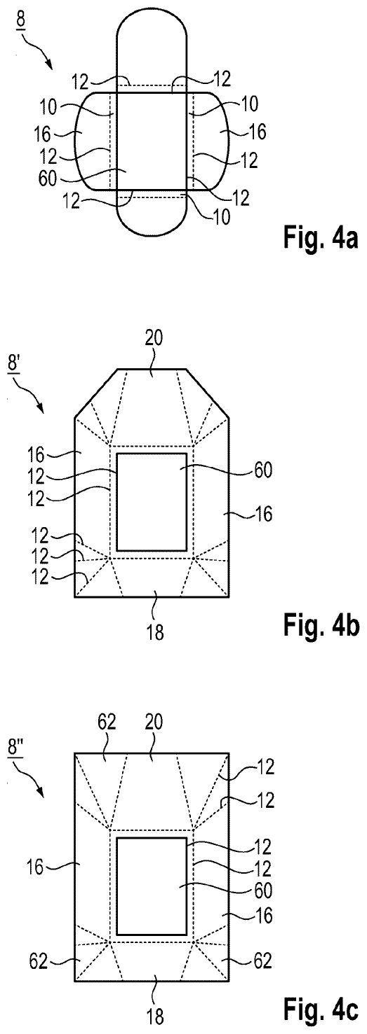

FIG. 4 shows some variants of a pre-cut wrapping blank 8 provided with folding edges 12, as could be used in the devices described above. FIG. 4a shows a wrapping blank 8 which could be used to form a wrapping 2 of the type and shape of an envelope. Here, the four inner folding edges 12 are arranged in a rectangle-like manner around the base area 60 of the envelope 2. An associated outer folding edge 12 is then provided, each offset outwards and parallel to the inner folding edge 12, so that the side surfaces or side flaps 10 of the envelope 2 are formed in each case between the inner and outer folding edges 12. A wrapping blank 8 designed in this way can be processed comparatively simply and reliably in the folding process described above, whereby the hygiene article to be packaged is wrapped in the wrapping 2 in a manner comparable to an envelope.

In a further developed wrapping concept, which is considered to be independently inventive, the wrapping blank 8 is, in contrast, also designed to completely enclose the hygiene article. In particular, it is taken into account that the wrapping blank 8 shown in FIG. 4a could leave openings to a small extent in the area of the forming corners after folding. This could make it more difficult to store products that are kept moist or, on the other hand, also hinder the dry storage of the hygiene products in a humid outdoor environment.

To counteract this, in the embodiments of the wrapping blank 8′, 8″ shown in FIG. 4b and FIG. 4c, which are considered to be independently inventive, the folding edges 12 are positioned in such a way that after folding in the forming wrapping 2, its corners are completely covered by independent segments of material. For this purpose, a number of inner folding edges 12 are also provided in these embodiments, which are arranged in a rectangle-like manner around the base area 60 of the wrapping 2. In these embodiments, however, there are a number of further segments of material 62 between the side flaps 16 and the respective adjacent front or rear flap 18, 20, which are folded over the forming corners during folding. Compared to common products, the resulting folding pattern corresponds approximately to that of a chocolate bar wrapper.

In these variants, the wrapping blank 8′, 8″ can be contoured and cut to size in different ways, starting from an initially rectangular pre-cut, depending on the individually specified purpose. FIG. 4b shows an example with a slightly cut upper area, whereby the resulting waste (i.e. the waste produced when cutting from a rectangular blank) can already be kept comparatively low. In contrast, a complete avoidance of waste is made possible by the wrapping blank 8″ as shown in FIG. 4c. In this case, the entire base area of the originally rectangular blank can be used to form the partial areas forming the wrapping 2.

### LIST OF REFERENCE SIGNS

1 hygiene product
2 wrapping
4, 6 arrow
8, 8′, 8″ wrapping blank
10 side edge
12 folding edge
14 arrow
15 print mark
16 side flap
18 front flap
20 rear flap
22 motif
24 window cut-out
30 material web
34 cutting unit
36 single sheet
40 wrapping unit
42 window element
50 material web
52 print mark
60 base area
62 material segment

1. A method for wrapping a hygiene article or a stack of a number of hygiene articles, namely absorbent disposable articles for absorbing body excretions, wherein the hygiene article or the stack of hygiene articles is provided with a wrapping formed from a sustainable, in particular biodegradable and/or recyclable, raw material from the group comprising paper, kraft paper, greaseproof and/or wet-strength paper, filter paper, crepe paper, glassine, eucalyptus, tissue, bamboo, papyrus, hemp, felt, wool, cotton, linen, in which
a. the hygiene article or the stack of hygiene articles is fed by means of a first feed unit to a wrapping station,
b. a wrapping blank formed from the sustainable raw material is provided in a pre-creasing station with folding edges suitable for forming a number of side edges or side faces of the wrapping and is then fed by means of a second feed unit to the wrapping station,
c. in the wrapping station, the hygiene article or the stack of hygiene articles is positioned in a predetermined desired position on the wrapping blank, and
d. in the wrapping station, the hygiene article or the stack of hygiene articles is wrapped by folding the wrapping blank and then closing it, by
d1. folding in the side flaps to form the side edges predefined by the folding edges,
d2. folding in the front and rear flaps to form the side edges predefined by the folding edges provided for them, and
d3. fixing the front and rear flaps to the side flaps and to one another,
wherein the wrapping blank is designed to completely enclose the hygiene article, and wherein there are a number of further segments of material between the side flaps and the respective adjacent front or rear flap which are folded over the forming corners during folding.

2. The method according to of claim 1, wherein the wrapping blank is separated from a continuous web of raw wrapping material before being fed to the wrapping station.

3. The method of claim 1, wherein the side edges of the wrapping blank are cut before or in the wrapping station.

4. The method of claim 1, wherein the flaps of the wrapping are fixed to each other thermally and/or mechanically and/or by gluing in or after the wrapping station.

5. The method of claim 1, in which the wrapping blank is provided with a printing.

6. The method of claim 1, in which the wrapping blank is provided with an embossing.

7. The method of claim 1, in which the wrapping blank is provided with a perforation or with perforations.

8. The method of claim 1, in which a window is cut out in the wrapping blank, which window is then closed by gluing in a window film.

9. The method of claim 1, in which the hygiene article or the stack of hygiene articles on the one hand and an associated wrapping blank on the other hand are fed to the wrapping station in a timed manner and synchronized with one another.

10. The method of claim 9, in which the timing or synchronization of hygiene articles or stack of hygiene articles and assigned wrapping blank is monitored by interrogating a print mark.

11. The method of claim 1, in which the raw material provided for forming the wrapping is multilayered.

12. The method of claim 1, wherein the hygiene article or articles are fixed to the wrapping after folding into the wrapping.

13. (canceled)
